(19) United States
(12) Patent Application Publication
(10) Pub. No.: US 2025/0367107 A1
(43) Pub. Date: Dec. 4, 2025

(54) SUNSCREEN COMPOSITION INCLUDING A SALICYLATE AND PONGAMIA PINNATA SEED EXTRACT

(71) Applicant: Edgewell Personal Care Brands, LLC, Chesterfield, MO (US)

(72) Inventors: Michael Russell, Madison, NJ (US); Sairohit Raghupathy, Lyndhurst, NJ (US)

(21) Appl. No.: 18/876,408

(22) PCT Filed: Jul. 28, 2023
(86) PCT No.: PCT/US2023/071225

§ 371 Date: Dec. 18, 2024

Related U.S. Application Data
(60) Provisional application No. 63/369,800, filed on Jul. 29, 2022.

(51) Int. Cl. A61K 8/9789; A61Q 17/04

(52) U.S. Cl. CPC A61K 8/9789; A61Q 17/04; A61K 2800/31

(57) ABSTRACT

A sunscreen composition including at least one organic sunscreen active and Pongamia pinnata Seed Extract. The at least one organic sunscreen active may be selected from at least one organic sunscreen active having no substantial photoprotection capabilities within the UVA-1 region. The at least one organic sunscreen active may be selected from a salicylate. For example, a salicylate may be present in an amount from about 3.0 wt. % to about 20.0 wt. % and the Pongamia pinnata Seed Extract may be present in an amount from 0.26 wt. % to 4.9 wt. %, based on the total weight of the sunscreen composition. Thus, for example, the weight ratio of homosalate to the Pongamia pinnata Seed Extract may be from about 9:1 to about 18:1 and the weight ratio of octisalate to the Pongamia pinnata Seed Extract may be from about 4.0:1 to about 9:1.

### CROSS-REFERENCE TO RELATED APPLICATIONS

This application claims priority to International Application PCT/US2023/071225, filed on Jul. 28, 2023, which claims priority to U.S. Provisional Patent Application No. 63/369,800, filed on Jul. 29, 2022, the contents of which are incorporated herein by reference in their entirety.

### TECHNICAL FIELD

Embodiments relate to a sunscreen composition including a synergistic combination of a salicylate and Pongamia pinnata Seed Extract for enhanced photoprotection within at least the Ultraviolet A-1 (UVA-1) region of the electromagnetic spectrum.

### BACKGROUND

Ultraviolet (UV) solar radiation falling within the UVA region (340-400 nm for UVA-1 and 320-340 nm for UVA-2) is damaging owing to its ability to reach deeper into the dermis layer. Meanwhile, UV solar radiation falling within the UVB region (290-320 nm) may be absorbed by the epidermis layer to cause erythema and cellular mutations that damage the skin or underlying tissue. Accordingly, sunscreen compositions include UVA and/or UVB sunscreen actives to protect keratinous substrates, such as skin and hair, from UV radiation.

Sunscreen compositions may also include boosters to enhance the photoprotection capability of the compositions. For example, Pongamia pinnata Seed Extract may boost the photoprotection capability of compositions having mineral sunscreen actives, such as titanium dioxide (TiO₂) and zinc oxide (ZnO), at relatively low extract use levels (0.01 wt. % to 0.25 wt. %). Nevertheless, sensory challenges such as unfavorable spreading or transparency remain for sunscreen compositions having only mineral sunscreen actives with boosters.

Meanwhile, preferable use levels for pongamol oil in personal care compositions may be relatively high such as 5 wt. % and up to 13 wt. %, 30 wt. %, or even 65 wt. %. In addition, the extract of Pongamia pinnata may necessarily be present with other required ingredients such as polyesters, extracts, or colorants. Alternatively, isolated pongamol or extract with selectively eliminated karanjin may be favored. Such personal care compositions may increase complexity or cost related to composition manufacture or use. Thus, there is room for improvement for a sunscreen composition including Pongamia pinnata Seed Extract.

### SUMMARY

Embodiments relate to a sunscreen composition including at least one organic sunscreen active and Pongamia pinnata Seed Extract. The sunscreen composition may be in any cosmetically acceptable form such as, without limitation, an emulsion, a gel, a lotion, a cream, a spray, a mist, a stick, a mouse, a foam, an ointment, a milk, a base, a concentrate, an anhydrous composition, an aqueous composition, or combinations thereof. The at least one organic sunscreen active may include one or more sunscreen actives selected from a p-aminobenzoic acid, a benzophenone, a camphor, a cinnamate, a dibenzoylmethane, an imidazole, a triazole, a triazine, a triazole, a salicylate, a metal oxide, or combinations thereof.

The at least one organic sunscreen active may be selected from at least one organic sunscreen active having no substantial photoprotection capabilities within the Ultraviolet A-1 (UVA-1) region of the electromagnetic spectrum. Preferably, the at least one organic sunscreen active is selected from a salicylate. The salicylate may, for example, be selected from homosalate, octisalate, or combinations thereof. The salicylate may, for example, be present in an amount from about 1.0 wt. % to about 25.0 wt. %, such as from about 3.0 wt. % to about 20.0 wt. %, based on the total weight of the sunscreen composition.

In any of the preceding embodiments, the Pongamia pinnata Seed Extract may be present in an amount from about 0.2 to about 5.0, such as from 0.26 wt. % to 4.9 wt. %, based on the total weight of the sunscreen composition. In one example, the salicylate may include homosalate present in an amount of about 9.0 wt. % and the Pongamia pinnata Seed Extract may be present in an amount from 0.26 wt. % to 3.0 wt. %, based on the total weight of the sunscreen composition. In another example, the salicylate may include octisalate present in an amount of about 4.5 wt. % and the Pongamia pinnata Seed Extract may be present in an amount from 0.26 wt. % to 1.0 wt. %, based on the total weight of the sunscreen composition.

In any of the preceding embodiments, the ratio of salicylate to Pongamia pinnata Seed Extract may be from about 1:1 to about 100:1. For example, the ratio of salicylate to Pongamia pinnata Seed Extract may be from about 4:1 to about 20:1. The ratio of homosalate to Pongamia pinnata Seed Extract may, for example, be about 9:1 to about 18:1. The ratio of octisalate to Pongamia pinnata Seed Extract may, for example, be about 4.5:1 to about 9:1.

In any of the preceding embodiments, the sunscreen composition may include one or more auxiliary ingredients. Additionally or alternatively, Pongamia pinnata Seed Extract may include pongamol and karanjin, and the sunscreen composition may be free of isolated pongamol, isolated karanjin, tamanu oil, Camellia sinensis leaf extract, green coffee seed extract, Angelica archangelica root extract, citrus aurantium peel extract, mica, ruby, titanium dioxide, zinc oxide, polyesters, trimethoxybenzylidene pentanedione, penthaerythrityl tetra-di-t-butyl hydroxyhydrocinnamate, or combinations thereof.

Embodiments further relate to a consumer-packaged product comprising a container housing the sunscreen composition of any of the preceding embodiments. Embodiments may include using various packaged formats such as, without limitation, pump containers, spray containers, mist containers, tubes, jars, boxes, and the like. The container may be made of any suitable material such as, without limitation, plastic, compostable material, biodegradable material, metal, or combinations thereof.

Embodiments further relate to a method to protect a keratinous surface from UV radiation comprising contacting the keratinous surface with the sunscreen composition of any of the preceding embodiments. Embodiments further relate to a method to make the sunscreen composition of any of the preceding embodiments. The method may include identifying at least one organic sunscreen active having no substantial photoprotection capabilities within the UVA-1 region of the electromagnetic spectrum and mixing the Pongamia pinnata Seed Extract with the at least one organic sunscreen active for a synergistic increase in the sunscreen composition to at least the UVA-1 absorbance, the critical wavelength, the UVA-1/UV ratio, or combinations thereof.

### BRIEF DESCRIPTION OF THE DRAWINGS

The features of the disclosure believed to be novel and the elements characteristic of the invention are set forth with particularity in the appended claims. The figures are for illustration purposes only and are not drawn to scale. The disclosure itself, however, both as to organization and method of operation, can best be understood by reference to the description of the preferred embodiment(s) which follows, taken in conjunction with the accompanying drawings in which:

FIG. 1 is a graph showing a UV photoprotection synergistic effect in an anhydrous sunscreen composition including an organic UVB sunscreen active and Pongamia pinnata Seed Extract according to embodiments;

FIG. 2 is a graph showing a dose dependent UV photoprotection boost effect in an anhydrous sunscreen composition including an organic UVA-UVB sunscreen active and Pongamia pinnata Seed Extract according to embodiments;

FIG. 3 is a graph showing a UV photoprotection neutral effect in an anhydrous sunscreen composition including an organic UVA sunscreen active and Pongamia pinnata Seed Extract according to embodiments;

FIG. 4 is a graph showing a UV photoprotection synergistic effect in an aqueous sunscreen composition including an organic UVB sunscreen active and Pongamia pinnata Seed Extract according to embodiments;

FIG. 5 is a graph showing a dose dependent UV photoprotection boost effect in an aqueous sunscreen composition including an organic UVA-UVB sunscreen active and Pongamia pinnata Seed Extract according to embodiments; and

FIG. 6 is a graph showing a UV photoprotection neutral effect in an aqueous sunscreen composition including an organic UVA sunscreen active and Pongamia pinnata Seed Extract according to embodiments.

### DETAILED DESCRIPTION

Embodiments can comprise, consist of, and consist essentially of the features and/or steps described herein, as well as any of the additional or optional ingredients, components, steps, or features described herein or would otherwise be appreciated by one of skill in the art. It is to be understood that all concentrations disclosed herein are by weight percent (wt. %.) based on a total weight of the composition unless otherwise indicated. Where appropriate, the International Nomenclature of Cosmetic Ingredients (INCI) name of ingredients/components is provided. In this regard, the term “(and)” is an INCI designation fora mixture of listed components separated by the term. Any numerical range recited herein is intended to include all sub-ranges subsumed therein, and such ranges are understood to include each and every number and/or fraction between the stated range lower and upper values. Moreover, the term “about” may refer to deviations of ±20%.

### Photoprotection Booster

A sunscreen composition includes a photoprotection booster to enhance the photoprotection capability of the sunscreen composition. The photoprotection booster preferably includes extract of the seeds of Pongamia pinnata, also known as Pongamia glabra, Millettia pinnata, Derris indica and Gadelupa pinnata, which is a monotypic genus indigenous to the Indian subcontinent and south-east Asia. Pongamia pinnata Seed Extract may include relatively low levels of 1,3-propanedione 1-(4-methoxy-5-benzofuranyl)-3 phenyl, a diketone known as pongamol (CAS #: 484-33-3), and relatively low levels of (3-methoxy-2-phenylfuro[2,3-h]chromen-4-one), a furanoflavonol known as karanjin (CAS #: 521-88-0). Pongamia pinnata Seed Extract is readily commercially available, such as extract sold under the trade name ASSURE+™ (EverCare®, EverZinc UltraFine Products Inc.).

As discussed in further detail below, an unexpected synergy in photoprotection within at least the UVA-1 region of the electromagnetic spectrum is observed in a sunscreen composition composed of Pongamia pinnata Seed Extract and an organic sunscreen active that traditionally has no substantial photoprotection capabilities in the UVA-1 region of the electromagnetic spectrum. For example, an unexpected synergy in UVA-1 photoprotection is observed in a sunscreen composition composed of Pongamia pinnata Seed Extract and a salicylate selected from octisalate, homosalate, or combinations thereof. Moreover, there is no substantial detriment to photoprotection within the UVA region from a sunscreen composition composed of Pongamia pinnata Seed Extract and sunscreen actives that traditionally do have photoprotection capabilities in the UVA-1 region such as butyl methoxy dibenzoylmethane and bis-ethylhexyloxyphenol methoxyphenyl triazine.

Accordingly, relatively less organic sunscreen actives may be used to provide photoprotection at least in the UVA-1 region of the electromagnetic spectrum relative to sunscreen compositions without Pongamia pinnata Seed Extract. Additionally, the synergistic and unexpected effects are reproducible across hydrous formulation formats and anhydrous formulation formats. In addition, a sunscreen composition including Pongamia pinnata Seed Extract can be manufactured on a commercial scale and Pongamia pinnata Seed Extract can be compatible with various traditional sunscreen ingredients as evidenced from its incorporation into commercially available sunscreen products.

Sunscreen compositions according to embodiments also do not require auxiliary ingredients to impart synergistic and unexpected effects observed when in combination with certain organic sunscreen actives. For example, a sunscreen composition may be free (e.g., 1 wt. %, preferably at most trace amounts when present) of certain isolated components extracted from Pongamia pinnata, certain skin care ingredients, additional boosters, colorant powders, mineral sunscreen actives, photostabilizers, or combinations thereof. Sunscreen compositions including Pongamia pinnata Seed Extract may, for example, be free of one or more of the following: isolated pongamol, isolated karanjin, tamanu oil, Camellia sinensis leaf extract, green coffee seed extract, Angelica archangelica root extract, citrus aurantium peel extract, mica, ruby, titanium dioxide, zinc oxide, polyesters, trimethoxybenzylidene pentanedione, and/or penthaerythrityl tetra-di-t-butyl hydroxyhydrocinnamate.

Notwithstanding the above, sunscreen compositions according to embodiments may optionally include any auxiliary ingredients such as, for example, isolated components of Pongamia pinnata, any skin care ingredients, additional boosters, colorant powders, mineral sunscreen actives, photostabilizers, or combinations thereof. For example, sunscreen compositions according to embodiments may include traditional boosters such as, without limitation, polysaccharide boosters (e.g., carboxymethylxyloglucan, galactoarabinan, etc.), cellulose boosters (e.g., methylcellulose, hydroxypropyl methylcellulose, microcrystalline cellulose, etc.), starch boosters (e.g., maize starch, potato starch, wheat starch, rice starch, cassava starch, tapioca starch, barley starch, etc.), or combinations thereof. Thus, auxiliary ingredients discussed herein as well alternatives, modifications, and variations falling within the true scope of the present disclosure, such as synthetic boosters or photostabilizers, are contemplated in aspects of embodiments.

A photoprotection booster may be present in an amount from about 0.1 wt. % to about 10.0 wt. %, based on the total weight of the sunscreen composition. For example, one or more photoprotection boosters may be present in an amount of 0.08 wt. %, 1.0 wt. %, 5.0 wt. %, 10.0 wt. %, 12.0 wt. %, including every number and/or fraction therebetween, based on the total weight of the sunscreen composition. Thus, for example, Pongamia pinnata Seed Extract may be present in an amount from 0.26 wt. % to 4.9 wt. %, such as 0.26 wt. %, 0.3 wt. %, 0.35 wt. %, 0.4 wt. %, 0.45 wt. %, 0.5 wt. %, 0.55 wt. %, 0.6 wt. %, 0.65 wt. %, 0.7 wt. %, 0.75 wt. %, 0.8 wt. %, 0.85 wt. %, 0.9 wt. %, 0.95 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.5 wt. %, 3.0 wt. %, 4.0 wt. %, 4.5 wt. %, 4.9 wt. %, including every number and/or fraction therebetween, based on the total weight of the sunscreen composition.

### Sunscreen Active

A sunscreen composition includes a sunscreen active capable of substantially absorbing, scattering, and/or deflecting UV radiation on a keratinous substrate. Preferably, the sun protection factor (SPF) of a sunscreen composition is ≥8 and the UVA protection factor (UVAPF) is at least ⅓ the SPF. Thus, for example, physical or mineral sunscreen actives may be selected from metal oxides (e.g., titanium dioxide, zinc oxide, etc.) that can be used in effective amounts to provide an SPF of 8 or more (in contrast to other uses as colorants, etc.). Such actives may have various morphologies including crystalline form (e.g., rutile, etc.), size (e.g., 5 nm-1.0 μm, etc.), shape (e.g., amorphous, spherical, etc.), and/orfunctionalization (e.g., coatings, etc.) that allow for the effective use as sunscreen actives. For example, titanium dioxide may have a coating of silicone copolymers, fatty acids, salts of fatty acids, oxides of silicon, oxides of aluminum, oxides of zirconium, or combinations thereof. Such actives may also be dispersed in one or more dispersing agents such as, for example, alkyl benzoates, polyhydroxystearic acid, stearic acid, or combinations thereof.

Meanwhile, organic sunscreen actives may be selected from a p-aminobenzoic acid, a benzophenone, a camphor, a cinnamate, a dibenzoylmethane, a salicylate, an imidazole, a triazole, a triazine, a triazole, or combinations thereof. Organic sunscreen actives may include one or more of the following: p-aminobenzoic acids such as p-aminobenzoic acid (CAS #: 150-13-0), p-aminobenzoic acid, monoglyceryl ester (CAS #: 136-44-7), p-aminobenzoic acid, octyl dimethyl ester (padimate-0, CAS #: 21245-02-3), p-aminobenzoic acid, ethyl dihydroxypropyl (roxadimate, CAS #: 58882-17-0), p-aminobenzoic acid, 2,4-dihydroxy-N-(3-hydroxypropyl)-3,3-dimethylbutanamide ester, p-aminobenzoic acid, PEG-25 (CAS #: 116242-27-4); anthranilates such as menthyl anthranilate (CAS #: 134-09-8); benzophenones such as benzophenone (CAS #: 119-61-9), 2,4-dihydroxybenzophenone (benzophenone-1, CAS #: 131-56-6), 2,2′,4,4′-tetrahydroxybenzophenone (benzophenone-2, CAS #: 131-55-5), 2-hydroxy-4-methoxybenzophenone (benzophenone-3, oxybenzone, CAS #: 131-57-7), 2-hydroxy-4-methoxybenzophenone-5-sulfonic acid (benzophenone-4, sulisobenzone, CAS #: 4065-45-6), 2-hydroxy-4-methoxybenzophenone-5-sulfonic acid, monosodium salt (benzophenone-5, CAS #: 6628-37-1), 2,2′-dihydroxy-4,4′-dimethoxybenzophenone (benzophenone-6, CAS #: 131-54-4), 5-chloro-2-hydroxybenzophenone (benzophenone-7, CAS #: 85-19-8), 2,2′-dihydroxy-4-methoxybenzophenone (benzophenone-8, dioxybenzone, CAS #: 131-53-3), 2,2′-dihydroxy-4,4′-dimethoxybenzophenone-5,5′-disulfonic acid sodium salt (benzophenone-9, CAS #: 76656-36-5), 2-hydroxy-4-methoxy-4′-methyl-benzophenone (benzophenone-10, Mexenone, CAS #: 1641-17-4), bis(2,4-ihydroxyphenyl)methanone (benzophenone-11, CAS #: 1341-54-4), 2-hydroxy-4-(octyloxy)benzophenone (benzophenone-12, octabenzone, CAS #: 1843-05-6), 2,2′-dihydroxy-4-methoxybenzophenone (dioxybenzone, CAS #: 131-53-3), 2-hydroxy-4-methoxybenzophenone (oxybenzone, CAS #: 131-57-7), 2-hydroxy-4-methoxybenzophenone-5-sulphonic acid (sulisobenzone, CAS #: 4065-45-6), hexyl 2-(4-(diethylamino)-2-hydroxybenzoyl)benzoate (CAS #: 302776-68-7); camphors such as benzylidene camphor sulfonic acid (CAS #: 56039-58-8), 3-benzylidene camphor (CAS #: 15087-24-8), polyacrylamidomethyl benzylidene camphor (CAS #: 113783-61-2), terephthalylidene dicamphor sulfonic acid (CAS #: 90457-82-2), 3-(4-methylbenzyliden)camphor (CAS #: 36861-47-9), camphor benzalkonium methosulfate (CAS #: 52793-97-2), bornelone (CAS #: 2226-11-1); cinnamates such as ethyl cinnamate (CAS #: 103-36-6), 2-ethoxyethyl-p-methoxycinnamate (cinoxate, CAS #: 104-28-9), isoamyl p-methoxycinnamate (CAS #: 71617-10-2), diisopropyl methyl cinnamate (CAS #: 32580-71-5), 2-ethylhexyl alpha-cyano-beta-phenylcinnamate (octocrylene, CAS #: 6197-30-4), diethanolamine methoxycinnamate (CAS #: 56265-46-4), isopropyl methoxycinnamate (CAS #: 5466-76-2), isoamyl p-methoxycinnamate (CAS #: 71617-10-2), glyceryl octanoate dimethoxycinnamate, ethyl diisopropylcinnamate (CAS #: 32580-72-6), ethyl methoxycinnamate (CAS #: 99880-64-5), octyl methoxycinnamate (octinoxate, CAS #: 5466-77-3); dibenzoylmethanes such as dibenzoylmethane (CAS #: 120-46-7), isopropyl dibenzoylmethane (eusolex, CAS #: 63250-25-9), 4-tert-Butyl-4′-methoxy-dibenzoylmethane (avobenzone, CAS #: 70356-09-1); salicylates such as 3,3,5-trimethylcyclohexyl salicylate (homosalate, CAS #: 118-56-9), benzyl salicylate (CAS #: 118-58-1), octyl salicylate (octisalate, CAS #: 118-60-5), 2-hydroxyethyl salicylate (CAS #: 87-28-5), menthyl salicylate (CAS #: 89-46-3), isopropylbenzyl salicylate (CAS #: 94134-93-7); imidazoles such as phenylbenzimidazole, phenylbenzimidazol-5-sulfonic acid (CAS #: 27503-81-7), phenylbenzimidazole tea sulfonate (CAS #: 73705-00-7), urocanic acid [4-imidazoleacrylic acid, CAS #: 104-98-3], ethyl urocanate (CAS #: 27538-35-8), bisymidazylate (CAS #: 180898-37-7), sodium phenylbenzimidazole sulfonate (CAS #: 5997-53-5); triazines such as bis(ethylhexyloxyphenol methoxyphenol) triazine (bemotrizinol, CAS #: 187393-00-6); triazoles such as 2-(2-hydroxy-5-methyl-phenyl)benzotriazole (drometrizole, CAS #: 2440-22-4); triazones such as bis-ethylhexyloxyphenol methoxyphenyl triazine (iscotrizinol, CAS #: 154702-15-5), ethylhexyl triazone (CAS #: 88122-99-0); petrolatum (CAS #: 8009-03-8), acrylates such as diurethane dimethacrylate (CAS #: 103597-45-1), ethyl 2-cyano-3,3-diphenylacrylate (etocrilene, CAS #: 5232-99-5); and/or siloxanes such as drometrizole trisiloxane (CAS #: 155633-54-8).

Accordingly, embodiments may include a sunscreen active that protects against UVA radiation such as avobenzone (e.g., Parasol® 1789), diethylamino hydroxybenzoyl hexyl benzoate (e.g., Uvinul® A Plus), terephthalylidene dicamphor sulfonic acid, bis-disulizole disodium, disodium phenyl dibenzimidazole tetrasulfonate, bis-diethylamino hydroxybenzoyl benzoate, bis-benzoxazolylphenyl ethylhexylamino triazine, or combinations thereof. Additionally or alternatively, embodiments may include a sunscreen active that protects against UVB radiation such as octocrylene, octinoxate, octisalate, homosalate, ensulizole, ethylhexyl triazone (e.g., Uvinul® T150), enzacamene, amiloxate, bemotrizinol (e.g., Uvasorb® HEB), benzylidene malonate polysiloxane, padimate-O, trolamine salicylate, cinoxate, PABA, or combinations thereof. Additionally or alternatively, embodiments may include an organic sunscreen active that protects against UVA and UVB radiation such as oxybenzone, meradimate, bis-octrizole, bis-ethylhexyloxyphenol methoxyphenyl triazine (Tinosorb® S), drometrizole trisiloxane, sulisobenzone, dioxybenzone, or combinations thereof. Additionally or alternatively, embodiments may include a sunscreen active for protection against UVA, UVB, VIS, and IR radiation such as titanium dioxide, zinc oxide, or combinations thereof.

Preferably, sunscreen compositions according to embodiments include at least one organic sunscreen active traditionally without substantial photoprotection capabilities in the UVA-1 region of the electromagnetic spectrum. For example, a sunscreen composition preferably includes a salicylate such as, without limitation, octisalate, homosalate, or combinations thereof. Sunscreen compositions according to embodiments may, however, include one or more sunscreen actives having photoprotection capabilities in the UVA and/or UVB region of the electromagnetic spectrum. For example, a sunscreen composition may include butyl methoxy dibenzoylmethane, bis-ethylhexyloxyphenol methoxyphenyl triazine, zinc oxide, titanium dioxide, or combination thereof.

A sunscreen active may be present in an amount from about 1.0 wt. % to about 40.0 wt. %, based on the total weight of the sunscreen composition. For example, one or more sunscreen actives may be present in an amount of 0.8 wt. %, 1.0 wt. %, 5.0 wt. %, 10.0 wt. %, 15.0 wt. %, 20.0 wt. %, 30.0 wt. %, 40.0 wt. %, 44.0 wt. %, including every number and/or fraction therebetween, based on the total weight of the sunscreen composition. Thus, a salicylate (e.g., homosalate, octisalate, etc.) may, for example, be present in an amount up to about 25.0 wt. %, such as 1.0 wt. %, 2.0 wt. %, 3.0 wt. %, 4.0 wt. %, 4.5 wt. %, 5.0 wt. %, 6.0 wt. %, 7.0 wt. %, 8.0 wt. %, 9.0 wt. %, 10.0 wt. %, 11.0 wt. %, 12.0 wt. %, 13.0 wt. %, 14.0 wt. %, 15.0 wt. %, 16.0 wt. %, 17.0 wt. %, 18.0 wt. %, 19.0 wt. %, 20.0 wt. %, 21.0 wt. %, 22.0 wt. %, 23.0 wt. %, 24.0 wt. %, 25.0 wt. %, 30.0 wt. %, including every number and/or fraction therebetween, based on the total weight of the sunscreen composition.

### Auxiliary Ingredient

A sunscreen composition may include auxiliary ingredients such as, without limitation, one or more of the following: carriers, film formers, surfactants, anti-aging actives, insect repellants, amino acids, colorants, pigments, dyes, opacifiers, pH adjusters, preservatives, preservative boosters, chelating agents, stabilizers, sphingolipids, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, skin cooling compounds, skin pollution protectants, skin penetration enhancers, exfoliants, staining agents, depigmenting agents, hypopigmenting agents, vitamins, antioxidants, alpha and beta hydroxy acids, retinol and its derivatives, propellants, or combinations thereof.

An auxiliary ingredient may be present in an amount of about 0.001 wt. % to about 99.8 wt. %, based on the total weight of the skin care composition. For example, one or more auxiliary ingredients may be present in an amount of 0.0008 wt. %, 0.008 wt. %, 0.01 wt. %, 0.1 wt. %, 0.25 wt. %, 0.5 wt. %, 1.0 wt. %, 2.5 wt. %, 5.0 wt. %, 10.0 wt. %, 20.0 wt. %, 40.0 wt. %, 60.0 wt. %, 90.0 wt. %, 99.8 wt. %, including every number and/or fraction therebetween, based on the total weight of the sunscreen composition.

Accordingly, for example, sunscreen compositions may be composed of one or more carriers including, without limitation, water, vegetable oils, esters (e.g., octyl palmitate, isopropyl myristate, isopropyl palmitate, etc.), ethers (e.g., dicapryl ether, dimethyl isosorbide, etc.), C₂-C₄ alcohol (e.g., ethanol, isopropanol, etc.), fatty alcohols (e.g., cetyl alcohol, stearyl alcohol, behenyl alcohol, etc.), isoparaffins (e.g., isooctane, isododecane, isohexadecane, etc.), silicones (e.g., dimethicones, polysiloxanes, etc.), hydrocarbon oils (e.g., mineral oil, petrolatum, isoeicosane polyisobutene, etc.), polyols (e.g., propylene glycol, glycerin, butylene glycol, pentylene glycol, hexylene glycol, etc.), certain salicylates, adipates, aliphatic molecules (e.g., micelles, oleosomes, liposomes, etc.), or combinations thereof.

For example, sunscreen compositions may be composed of one or more carriers that act as skin-conditioning agents (e.g., emollients, humectants, etc.). Examples of useful skin-conditioning agents include, without limitation, esters (e.g., isopropyl myristate, isopropyl palmitate, 1-decene polymer (hydrogenated), esters of benzoic acid such as lauryl/myristyl benzoate, C₁₂-C₁₅ alkyl benzoate, C₁₆-C₁₇ alkyl benzoate, stearyl benzoate, isostearyl benzoate, behenyl benzoate, ethylhexyl benzoate, butyloctyl benzoate, hexyldecyl benzoate, octyldodecyl benzoate, etc.), polyols (e.g., glycerin, dipropylene glycol, hexylene glycol, 2,3-butanediol, 1,10-decanediol, propylene glycol, 1,2-butanediol, pentylene glycol, ethyl hexanediol, caprylyl glycol, decylene glycol, butylene glycol, propanediol, etc.), natural oils (e.g., nut oil, seed oil, fruit oil, etc.), fatty acids (e.g., lanolin, petrolatum, mineral oil, 10-hydroxydecanoic acid, etc.), triglycerides (caprylic/capric triglyceride, etc.), silicones (cyclomethicone, dimethicone, etc.), certain salicylates (e.g., butyloctyl salicylate, C₁₂-C₁₅ alkyl salicylate, capryloyl salicylic acid, hexydodecyl salicylate, isocetyl salicylate, isodecyl salicylate, tridecyl salicylate, etc.), adipates (e.g., diisopropyl adipate, etc.) isoparaffins (e.g., isohexadecane, isododecane, etc.), amino acids and derivatives or salts thereof (e.g., sodium pyrrolidone carboxylic acid, etc.), oleosomes (oil bodies), liposomes, or combinations thereof.

Water may also be intentionally added in skin care compositions at an amount greater than 15.0 wt. % such as, for example, 25.0 wt. %, 45.0 wt. %, 58.0 wt. %, 65.0 wt. %, 75.0 wt. %, including every number and/or fraction therebetween, based on the total weight of a hydrous sunscreen composition. For example, a water-in-oil (W/O) emulsion may have an aqueous dispersed phase stabilized by an emulsifier and disposed in a hydrophobic continuous phase. Water may, however, be intentionally added in an amount of 15.0 wt. % or less, based on a total weight of the sunscreen composition, to form substantially anhydrous sunscreen compositions. Moreover, one or more ingredients may have associated with it absorbed and/or adsorbed water that can be carried into the otherwise substantially anhydrous sunscreen compositions. For economic, sourcing, and/or formulation reasons, it may be disadvantageous to process hygroscopic materials to make them completely free of water. Thus, substantially anhydrous sunscreen compositions may include 15.0 wt. % or less, preferably 10.0 wt. % or less and more preferably 5.0 wt. % or less (e.g., 1.0 wt. %), of water based on a total weight of the sunscreen composition.

A carrier may be present in an amount from about 0.01 wt. % to about 99.0 wt. %, based on the total weight of the skin care composition. For example, one or more carriers may be present in an amount of 0.008 wt. %, 0.01 wt. %, 0.1 wt. %, 1.0 wt. %, 5.0 wt. %, 10.0 wt. %, 15.0 wt. %, 20.0 wt. %, 60.0 wt. %, 90.0 wt. %, 99.0 wt. %, including every number and/or fraction therebetween, based on the total weight of the sunscreen composition.

Film former polymers may be capable of providing a substantially homogenous and/or structured hydro-lipidic film on skin. Generally, film former polymers may be synthesized from two or more different monomers to form copolymers and/or may from individual polymer chains that are connected by bridging molecules or crosslinking agents to form crosslinked copolymers known as cross polymers. For example, film former polymers may include monomers, copolymers, crosspolymers, and/or terpolymers of an organo-silicone hybrid, (meth)acrylate, abietic acid derivative, olefin, silicone, resin, vinyl acetate (VA), vinyl pyrrolidone (VP), maleate, alkyl ester, long chain or short chain carboxylic acid, or combinations thereof. For example, polymers used to form films may have a molecular weight (MW) of about 1,500,000 Daltons (DA) or less.

Film former polymers may include, for example, crotonic acid/vinyl C₈-C₁₂ isoalkyl esters/VA/bis-vinyldimethicone crosspolymer, acrylate/dimethicone copolymer, VA/butyl maleate/isobornyl acrylate copolymer, acrylates/C₁₂₋₂₂ alkyl methacrylate copolymer, acrylates/octylacrylamide copolymer, methyl dihydroabietate, trimethylsiloxysilicate, C₂₄-C₂₈ alkyldimethylsiloxy trimethylsiloxysilicate, polymethylsilsesquioxane/trimethylsiloxysilicate, polymethylsilsesquioxane, perfluorononyl dimethicone, trifluoropropyldimethylsiloxy/trimethylsiloxy silsesquioxane, PPG-17/isophorone diisocyanate/dimethylol propionic acid copolymer, polyurethane (e.g., 32, 34, 35, 48, etc.), hydrolyzed wheat protein/PVP crosspolymers, maltodextrin/VP copolymer, butylated PVP copolymer, VP/polycarbamylpolyglycol ester, VP/dimethylaminoethylmethacrylate/polycarbamyl polyglycol ester, VP/dimethiconyl-acrylate/polycarbamyl polyglycol ester, VP/eicosene copolymer, adipic acid/diglycol crosspolymer, trimethylpentanediol/adipic acid/glycerin crosspolymer, polyester-7 (and) neopentyl glycol diheptanoate, polyester-10 (and) propylene glycol dibenzoate, hydrogenated polycyclopentadiene (and) isododecane, stearyl/octyldodecyl citrate crosspolymer, hydroxyethyl acrylate/sodium acryloyldimethyl taurate copolymer, polysilicone-11, or combinations thereof.

A film former polymer may be present in an amount from about 0.1 wt. % to about 5.0 wt. %, based on the total weight of the skin care composition. For example, one or more film former polymers may be present in an amount of 0.08 wt. %, 0.5 wt. %, 1.0 wt. %, 2.0 wt. %, 2.5 wt. %, 3.5 wt. %, 4.0 wt. %, 5.0 wt. %, 6.0 wt. %, including every number and/or fraction therebetween, based on the total weight of the sunscreen composition.

Surfactants may include anionic, cationic, nonionic, amphoteric, or combinations thereof. Examples of useful anionic surfactants include, without limitation, carboxylates, amino acid derivatives, alkyl sulphates, alkyl ether sulfates, sulphonates, isethionates, taurates, sulfosuccinates, alkyl sulfoacetates, phosphates, alkyl phosphates, or combinations thereof. Examples of useful cationic surfactants include, without limitation, alkylamines, alkylimidazolines, quaternary ammonium compounds (QAC), or combinations thereof. Examples of useful nonionic surfactants include, without limitation, alkanolamides, amine oxides, esterified carboxylic acids, ethoxylated alcohols, poloxamers, or combinations thereof. Examples of useful amphoteric/zwitterionic surfactants include, without limitation, betaines, sultaines, cocamphoacetates, or combinations thereof.

Examples of emulsifiers, which are surfactants that can stabilize emulsions, may include oil-in-water (O/W) emulsifiers, water-in-oil (W/O) emulsifiers, or combinations thereof. An O/W emulsifier may have a hydrophilic-lipophilic balance (HLB)≥7, for example 8 to 18. Examples of useful O/W emulsifiers include, without limitation, certain sucrose esters (e.g., sucrose stearate, sucrose laurate, etc.), sorbitan esters (e.g., sorbitan laurate, etc.), polyglyceryl esters (e.g., polyglyceryl-3 methylglucose distearate, etc.), ethoxylated fatty ethers and ethoxylated fatty esters having a HLB≥7, or combinations thereof. Examples of useful natural O/W emulsifiers include, without limitation, cetearyl alcohol (and) cetearyl glucoside, glyceryl stearate (and) cetearyl alcohol (and) sodium stearoyl lactylate, cetearyl wheat straw glycosides (and) cetearyl alcohol, cetearyl olivate (and) sorbitan olivate, or combinations thereof. Thus, a skin care composition may include O/W emulsifiers such as glyceryl stearate, PEG-100 stearate, cetearyl alcohol (and) coco-glucoside, cetearyl alcohol (and) dicetyl phosphate (and) ceteth-10 phosphate, polysorbate 80, tribehenin PEG-20 esters, decyl glucoside, or combinations thereof.

Meanwhile, a W/O emulsifier may have a HLB of <7, for example from 4 to 6. Examples of W/O emulsifiers include, without limitation, certain sucrose esters (e.g., sucrose distearate, etc.), sorbitan esters (e.g., sorbitan oleate, sorbitan dioleate, sorbitan stearate, etc.), polyglyceryl esters (e.g., polyglyceryl-4 isostearate, polyglyceryl dimerate isostearate, etc.), silicones (e.g., a copolymer of a silicone, an alkoxylated derivative of a silicone, or combinations thereof, such as cetyl polyethylene glycol (PEG)/polypropylene glycol (PPG)-10/1 dimethicone, lauryl PEG-8 dimethicone, etc.), fatty acid esters (e.g., glyceryl stearate, etc.), or combinations thereof. Examples of useful natural W/O emulsifiers include cetearyl olivate. Thus, a skin care composition may include W/O emulsifiers such as lauryl PEG-8 dimethicone, cetyl PEG/PPG-10/1 dimethicone, or combinations thereof. It is, however, understood that other emulsion formats may similarly be used such as, without limitation, oil-in-oil (0/0) or oil-in-silicon (O/S) emulsions (e.g., castor oil in silicone oil, etc.) to deliver ingredients (e.g., anti-acne agents, sunscreen filters, antioxidants, etc.) to a substance.

A surfactant may be present in an amount from about 0.1 wt. % to about 10.0 wt. %, based on the total weight of the skin care composition. For example, one or more surfactants may be present in an amount of 0.08 wt. %, 0.1 wt. %, 1.0 wt. %, 2.0 wt. %, 3.0 wt. %, 4.0 wt. %, 5.0 wt. %, 6.0 wt. %, 7.0 wt. %, 8.0 wt. %, 9.0 wt. %, 12.0 wt. %, including every number and/or fraction therebetween, based on the total weight of the sunscreen composition.

Thickeners may alter viscosity. Thickeners may include plant-based thickeners, mineral thickeners, wax thickeners, ionic thickeners, synthetic polymer thickeners, or combinations thereof. Plant-based polymer thickeners may include, without limitation, xanthan gum, konjac gum, guar gum, acacia gum, acacia senegal gum, cellulose derivatives such as hydroxycellulose, hydroxyethyl cellulose, or combinations thereof. Mineral thickeners may include, without limitation, silica, bentonite, magnesium aluminum silicate, or combinations thereof. Natural or synthetic waxes may include, without limitation, animal waxes, vegetable waxes, mineral waxes, petroleum waxes, silicone waxes, or combinations thereof. Waxes with a melt point from about 50° C. to 100° C. may act as setting agents to solidify a formula. Ionic thickeners may include, without limitation, halide thickeners such as magnesium chloride, sodium chloride, potassium chloride, zinc chloride, or combinations.

Synthetic thickeners may include crosslinked polymers such as alkyl acrylate polymers in which the co-monomers consist of at least one of the following: acrylic acid, sodium acrylate, methacrylic acid, or alkyl acrylate. Polymers consisting purely of acrylic acid are often referred to as carbomers. Thus, for example, single molecules of carbomers can be crosslinked to provide a crosspolymer with a MW over 1 billion DA. In one example, acrylic acid may be crosslinked with allyl sucrose or allyl pentaerythritol. Similarly, a copolymer of C₁₀₋₃₀ alkyl acrylates and one or more monomers of acrylic acid, methacrylic acid or one of their simple esters may be crosslinked (e.g., with an allyl (2-propenyl) ether of sucrose or an allyl ether of pentaerythritol) to form acrylates/C₁₀₋₃₀ alkyl acrylate crosspolymer. Other synthetic thickeners may include acrylate copolymers and crosspolymers such as, without limitation, sodium acrylate/sodium acryloyldimethyl taurate copolymer, polyacrylate crosspolymer-6, or combinations thereof.

A thickener may be present in an amount from about 0.001 wt. % to about 40.0 wt. %, based on the total weight of the sunscreen composition. For example, one or more plant-based, mineral, synthetic polymer, or ionic thickener may be present in an amount of 0.002 wt. %, 0.008 wt. %, 0.01 wt. %, 0.05 wt. %, 0.1 wt. %, 0.5 wt. %, 1.0 wt. %, 2.0 wt. %, 3.0 wt. %, 3.5 wt. %, 4.0 wt. %, including every number and/or fraction therebetween, based on the total weight of the sunscreen composition. Meanwhile, wax thickeners (solidifying agents) may be present in an amount up to about 40.0 wt. % such as, for example, 1.0 wt. %, 2.0 wt. 25%, 5.0 wt. %, 18.0 wt. %, 25.0 wt. %, 44.0 wt. %, including every number and/or fraction therebetween, based in the total weight of the sunscreen composition.

Antioxidants may be obtained, for example, from the extracts of flowers, fruits, vegetables, nuts, berries, plants, or combinations thereof. Antioxidants may include polyphenols and catechin derivatives such as catechin, epigallocatechin, epicatechin, epicatechin gallate, epigallocatechin gallate, gallocatechin gallate, gallocatechin, or combinations thereof. Such constituents are effective scavengers of reactive oxygen species and may also function indirectly as antioxidants through their effects on transcription factors and enzyme activities. Additionally or alternatively, antioxidants may include carotenoids (e.g., beta-carotene, lycopene, lutein, zeaxanthin), additional polyphenols (e.g., resveratrol), selenium, vitamins (A, B, C, E), grape seed extract, green tea extract, pomegranate, chamomile, Coenzyme Q10, derivatives thereof, or combinations thereof.

An antioxidant may be present from about 0.001 wt. % to about 5.0 wt. %, based on the total weight of the sunscreen composition. For example, one or more antioxidants may be present in an amount of 0.005 wt. %, 0.01 wt. %, 0.045, 0.098, 0.15 wt. %, 0.2 wt. %, 0.3 wt. %, 0.5 wt. %, 0.99 wt. %, 1.0 wt. %, 2.0 wt. %, 3.0 wt. %, 6.0 wt. %, including every number and/or fraction therebetween, based on the total weight of the sunscreen composition.

Skin care actives that provide skin benefits may include, without limitation, any of the traditional above-mentioned skin ingredients. For example, anti-acne actives may include, without limitation, benzoyl peroxide, salicylic acid, retinoids (e.g., adapalene, tretinoin, etc.), azelaic acid, antibiotics (clindamycin, erythromycin, sulfacetamide, etc.), sulfur ingredients, or combinations thereof. Anti-aging ingredients may include, without limitation, retinol, bakuchiol, or combinations thereof. For example, bakuchiol may improve skin tone, improve skin elasticity, smooth fine lines, smooth wrinkles, or combinations thereof. Depigmenting agents may include, without limitation, lactic acid, niacinamide, arbutin, kojic acid, ferulic acid, hydroquinone, resorcinol, tyrosinase inhibitor, derivatives thereof, or combinations thereof. For example, vitamin B3 or derivative thereof may include niacinamide, nicotinic acid esters, non-vasodilating esters of nicotinic acid, nicotinyl amino acids, nicotinyl alcohol esters of carboxylic acids, nicotinic acid N-oxide, niacinamide N-oxide, or combinations thereof.

A skin care active may be present from about 0.1 wt. % to about 10.0 wt. %, based on the total weight of the sunscreen composition. For example, one or more skin care actives may be present in an amount of 0.08 wt. %, 0.5 wt. %, 1.0 wt. %, 2.0 wt. %, 3.0 wt. %, 4.0 wt. %, 5.0 wt. %, 10.0 wt. %, 12.0 wt. %, including every number and/or fraction therebetween, based on the total weight of the sunscreen composition.

### Formulation and Product Format

Sunscreen compositions may include one or more formulation formats such as, without limitation, an emulsion (0/W, W/O, O/0, O/S, multiple emulsion, etc.), a gel, a lotion, a cream, a spray, a mist, a stick, a mouse, a foam, an ointment, a milk, a base, a concentrate, or combinations thereof. Such formats may initially or finally be substantially aqueous or substantially anhydrous. For example, a substantially anhydrous base may be used in an aqueous emulsion to form a lotion or a cream. Similarly, ingredients and use levels to make such formats may be readily adjusted and mixed in a single phase or in multiple phases to provide a desired commercial sunscreen product. Thus, for example, water content, alcohol content, oil content, emulsifiers, foaming agents, gelling agents, etc., may be included, omitted, adjusted, or mixed in a single phase or in multiple phases to provide a commercial sunscreen product that accounts for cost, aesthetics, and/or product performance objectives.

Moreover, sunscreen compositions can be filled into any number of bottles, tottles, tubes, etc., having a variety of closing structures, pumps, sprays (e.g., mists, aerosols, bag on valve), amongst others, or combinations thereof. A bag-on-valve system may, for example, be used to deliver a sunscreen composition using compressed gas such as air, nitrogen, or carbon dioxide. In addition, sunscreen compositions may be formulated as a concentrate which, when combined with a propellant, forms a continuous aerosol spray commercial product. Propellants may include, for example, isobutene, butane, propane, dimethyl ether, methyl ether, tetraflouroethane, 1,1-difluoroethane, nitrogen, or combinations thereof. Propellants generally exist as equilibrium of vapor and liquid and can be either dissolved in or miscible with the composition. Ratios of propellant to the concentrate can be adjusted to account for cost, aesthetics, and product performance objectives. Thus, the concentrate and the propellant may be added to an aerosol container in a ratio of, based on weight, about 90:10 to about 60:40, for example about 75:25, about 70:30, and so on. In one example, the aerosol propellant may be present in an amount of about 10 wt. % to about 70 wt. %, based on the total weight of the sunscreen composition (e.g., final composition).

### Example Sunscreen Compositions

All equipment was cleaned and sanitized before batching. Ingredients were mixed in as phases or as a single phase according to conventional processes. For homosalate, octisalate, and octocrylene alone, the sunscreen actives were added to a 1:1 mix of ethanol and caprylic/capric triglycerides without a heating requirement. For avobenzone and bemotrizinol, and in all combinations with Pongamia pinnata Seed Extract, the susncreen actives and the extract were heated up to about 40° C. to 60° C. in caprylic/capric triglycerides until a clear solution was obtained, and ethanol was added once the oil phase was at room temperature (about 25° C.). No pH adjustment was required for anhydrous compositions.

A similar procedure was followed for making lotions. All equipment was cleaned and sanitized before batching. Oil-phase ingredients were mixed together and heated to about 60° C. to 80° C. at a rate between 300-2000 revolutions per minute (RPM). Water-phase ingredients were also mixed together and heated to about 60° C. to 80° C. at a rate between 300-2000 RPM. Once the oil-phase and water-phase were between about 60° C. to 80° C., the oil-phase was added to the water-phase and the bulk was homogenized. The pH of the batch was adjusted to between about 5.5 to 6.5 until the batch was uniform, and final mixing on sweep blade continued as the batch cooled to room temperature.

In vitro data was obtained using a Labsphere Ultraviolet Transmittance Analyzer (Model UV-2000 available from the Solar Light Company, Philadelphia, Pennsylvania). Samples of the sunscreen compositions were transferred by an adjustable pipette and uniformly applied to a Schonberg sand-blast PMMA plate (roughness 6 μm) by finger with a pre-saturated finger cot. The weight of samples followed the United States Food and Drug Administration (FDA) Critical Wavelength test method, and 0.75 mg/cm² of sample was measured for absorbance on the Labsphere Analyzer. The samples were spread on the plates by circular finger motions for about 30 seconds and then in horizontal and vertical motions until the product was uniformly spread. After application, the coated plate was dried for about 30 minutes. The sample plates were placed inside the Labsphere Analyzer.

Built-in software used was UV-2000 application Version 1.1.0.0, wherein E_{λ} is the Commission Internationale de l'Eclairage (CIE) erythemal spectral effectiveness, S_{λ} is designated as the solar spectral irradiance, and T_{λ} is the spectral transmittance of the sample as measured on the UV-2000. An average of nine readings for each PMMA plate, with three PMMA plates per sample, was recorded as the in-vitro absorbance value.

Table 1 shows example anhydrous sunscreen compositions according to embodiments. Sunscreen Sample Nos. 1-3 include homosalate in an amount of 9.0 wt. %, based on the total weight of the sunscreen composition, without Pongamia pinnata Seed Extract (Sample No. 1) and with Pongamia pinnata Seed Extract in an amount of 0.5 wt. % (Sunscreen Sample No. 2) or 1.0 wt. % (Sunscreen Sample No. 3), based on the total weight of the sunscreen composition. Sunscreen Samples Nos. 4-15 similarly include sunscreen actives with and without Pongamia pinnata Seed Extract, in the amounts listed in Table 1. Equal parts of ethanol and caprylic/capric triglyceride were used for Sunscreen Samples Nos. 1-15 and Reference Samples Nos. 16-17.

TABLE 1
Example Anhydrous Sunscreen Compositions

| | | Sample | Extract | UVA-1 | UVA-1/ | |
|---|---|---|---|---|---|---|
| Component (INCI) | | No. | (wt. %) | Abs. | UV | λc |
| UVB Active: | Homosalate, 9.0 wt. % | 1 | 0 | 0.01 | 0.004 | 325 |
| Carrier: | Ethanol and Caprylic/Capric | 2 | 0.50 | 12.02 | 0.298 | 367 |
| | Triglyceride, Q.S. to 100 | 3 | 1.00 | 21.87 | 0.391 | 371 |
| UVB Active: | Octisalate, 4.5 wt. % | 4 | 0 | 0.07 | 0.005 | 324 |
| Carrier: | Ethanol and Caprylic/Capric | 5 | 0.50 | 10.93 | 0.354 | 368 |
| | Triglyceride, Q.S. to 100 | 6 | 1.00 | 15.81 | 0.432 | 372 |
| UVA-UVB Active: | Octocrylene, 6.0 wt. % | 7 | 0 | 4.99 | 0.155 | 345 |
| Carrier: | Ethanol and Caprylic/Capric | 8 | 0.50 | 12.83 | 0.302 | 364 |
| | Triglyceride, Q.S. to 100 | 9 | 1.00 | 20.49 | 0.365 | 369 |
| UVA-UVB Active: | Bemotrizinol, 3.0 wt. % | 10 | 0 | 27.10 | 0.455 | 372 |
| Carrier: | Ethanol and Caprylic/Capric | 11 | 0.50 | 30.84 | 0.468 | 373 |
| | Triglyceride, Q.S. to 100 | 12 | 1.00 | 34.54 | 0.472 | 373 |
| UVA Active: | Avobenzone, 3.0 wt. % | 13 | 0 | 39.60 | 0.583 | 380 |
| Carrier: | Ethanol and Caprylic/Capric | 14 | 0.50 | 39.28 | 0.579 | 379 |
| | Triglyceride, Q.S. to 100 | 15 | 1.00 | 39.31 | 0.571 | 379 |
| Carrier: | Ethanol and Caprylic/Capric | 16 | 0.50 | 9.12 | 0.562 | 374 |
| | Triglyceride, Q.S. to 100 | 17 | 1.00 | 16.63 | 0.546 | 375 |

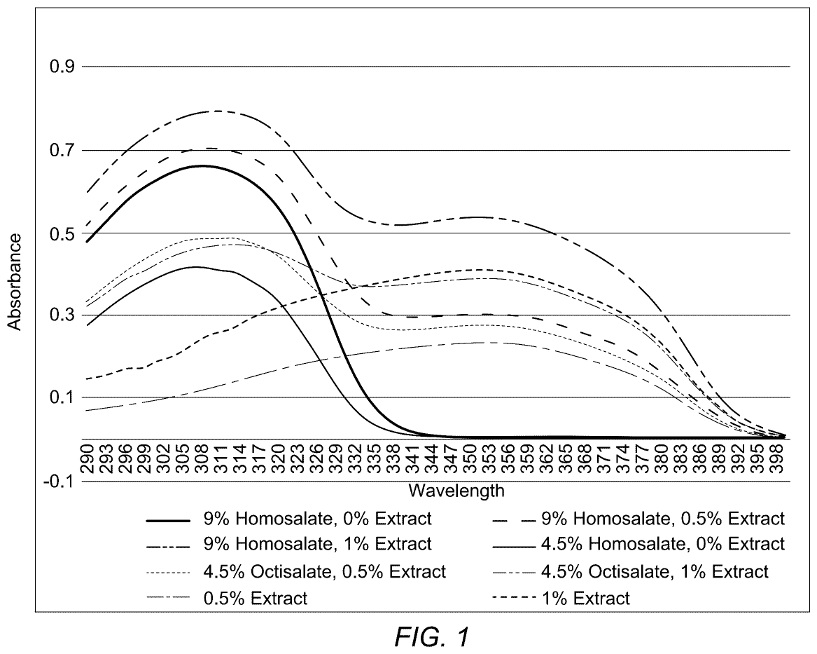

Referring to FIGS. 1-3, amplitude (vertical height) of the absorbance curves reflects the degree of photoprotection provided by the ingredients listed in Table 1. The higher the amplitude of the curve, the greater the absorbance, and the more protection provided at that wavelength. The greater the breadth of the absorbance curves, the broader the spectrum of sun protection provided. Sunscreen compositions that provide broad spectrum protection have broad (wide) absorbance curves that extend over a majority of the UV spectrum.

Referring now to FIG. 1, an unexpected synergy in UVA-1 photoprotection is observed in anhydrous sunscreen compositions including Pongamia pinnata Seed Extract and an organic sunscreen active that traditionally has no substantial photoprotection capabilities at least in the UVA-1 region. For example, the combined action from homosalate or octisalate with Pongamia pinnata Seed Extract on UVA-1 absorbance is greater than the sum of the individual effects on UVA-1 absorbance from the ingredients. As shown in Table 1, for example, UVA-1 absorbance of Sunscreen Sample No. 2 is 25% greater than the sum of UVA-1 absorbance from Sunscreen Sample No. 1 and Reference Sample No. 16. Extrapolation further indicates that UVA-1 absorbance may synergistically increase when Pongamia pinnata Seed Extract is present in an amount ≥0.26 wt. %, based on the total weight of the sunscreen composition. Also, Pongamia pinnata Seed Extract in an amount greater than 2.0 wt. % up to 4.9 wt. % may be useful for a synergistic increase with homosalate comparable to UVA-1 absorbance from abovenzone alone, while Pongamia pinnata Seed Extract in an amount 1.0 wt. % may be useful for a synergistic increase on UVA-1 absorbance with octisalate.

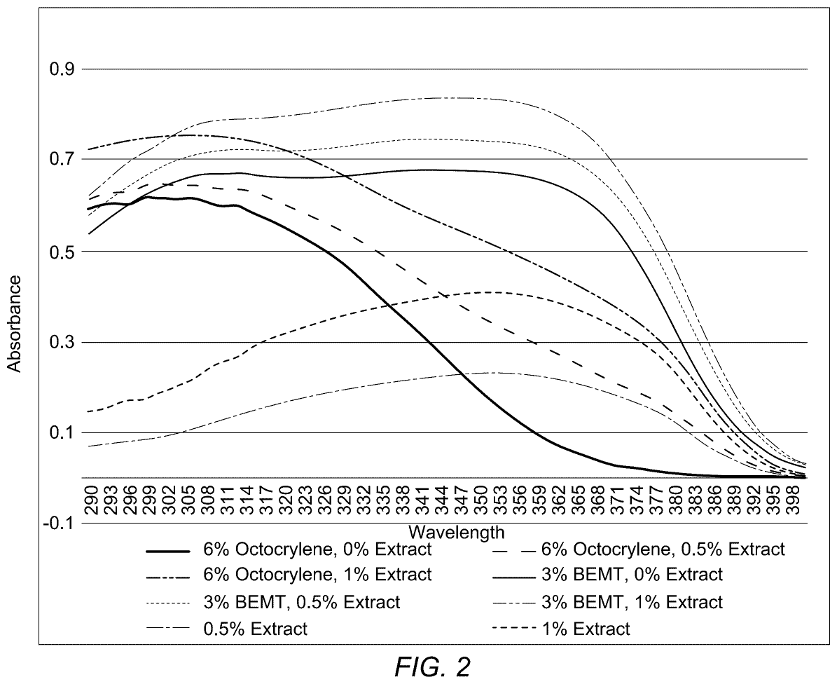

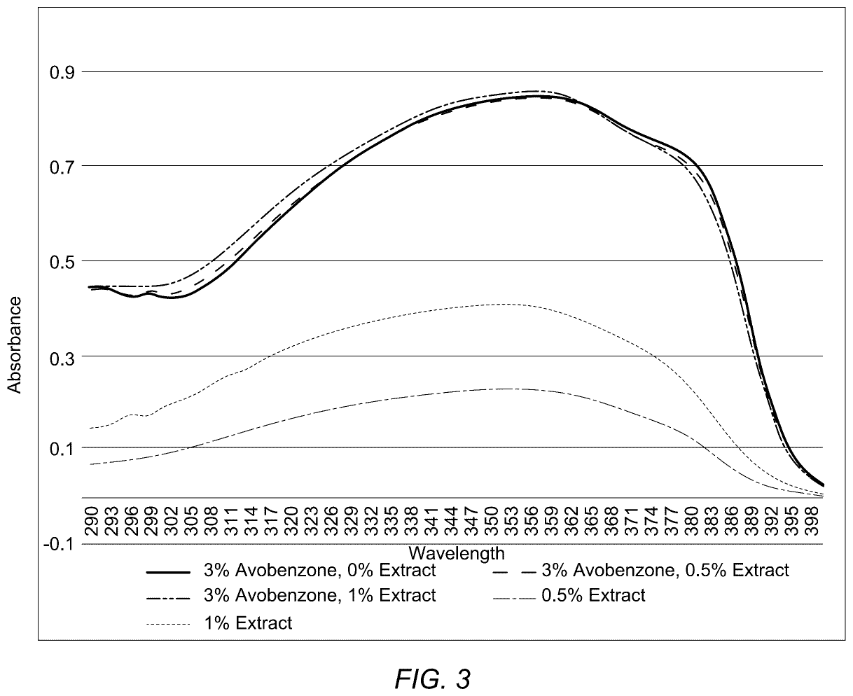

Turning now to FIG. 2, a dose dependent boost on UV photoprotection is observed in anhydrous sunscreen compositions including Pongamia pinnata Seed Extract and organic sunscreen actives that traditionally have photoprotection capabilities in both the UVA region and the UVB region. As shown in FIG. 2, the combined action from octocrylene or bemotrizinol with Pongamia pinnata Seed Extract provides a dose dependent boost on UV absorbance. This contrasts with the effects on photoprotection in anhydrous sunscreen compositions including Pongamia pinnata Seed Extract and organic sunscreen actives that traditionally have substantial photoprotection capabilities at least in the UVA region. For example, FIG. 3 shows an overall neutral effect in UVA photoprotection for the anhydrous sunscreen compositions including Pongamia pinnata Seed Extract and avobenzone. While no substantial boost on UVA photoprotection is observed in the anhydrous sunscreen compositions having avobenzone and Pongamia pinnata Seed Extract, no detriment to UVA photoprotection is also observed. Thus, various combinations of sunscreen actives may be incorporated at relatively lower levels for broad spectrum UV photoprotection.

Referring back to Table 1, mathematical integration of the measured absorbance from 290 nm to 400 nm was performed to calculate the area beneath the curve. The critical wavelength (λc), or the wavelength below which 90% of the area under the absorbance curve resides, was synergistically shifted in anhydrous sunscreen compositions including Pongamia pinnata Seed Extract and an organic sunscreen active that traditionally has no substantial photoprotection capabilities at least in the UVA-1 region. For example, the λc of Sunscreen Sample No. 2 was synergistically shifted from 349.5 to 367 and the λc of Sunscreen Sample No. 3 was synergistically shifted from 350 to 371. The unexpected higher λc values ensure more UV photoprotection, especially protection from longer wavelength UVA rays.

Meanwhile, substantially less dramatic shifts (if any) to the λc were observed in anhydrous sunscreen compositions including Pongamia pinnata Seed Extract and organic sunscreen actives that traditionally have photoprotection capabilities in both the UVA region and the UVB region such as octocrylene or bemotrizinol, while a relatively lower shift to the λc was observed in anhydrous sunscreen compositions including Pongamia pinnata Seed Extract and organic sunscreen actives that traditionally have substantial photoprotection capabilities at least in the UVA region of the electromagnetic spectrum such as avobenzone.

Using the FDA proposal to strengthen sunscreen broad spectrum requirements, spectral uniformity was also calculated based on the mean absorbance of UVA-1 (340-400 nm)/UV (290-400 nm). An unexpected synergy in the UVA-1/UV ratio is observed in anhydrous sunscreen compositions including Pongamia pinnata Seed Extract and an organic sunscreen active that traditionally has no substantial photoprotection capabilities at least in the UVA-1 region. For example, the combined action from homosalate or octisalate with Pongamia pinnata Seed Extract on UVA-1 absorbance is greater than the sum of the individual effects on UVA-1 absorbance from the ingredients. As shown in Table 1, for example, the UVA-1/UV ratio of Sunscreen Sample No. 5 is substantially greater (0.354) than the sum of UVA-1 absorbance from Sunscreen Sample No. 4 and Reference Sample No. 16 (0.284). Although less than the preferred threshold value of 0.7 for the UVA-1/UV ratio is illustrated, it is now apparent that this threshold can be readily obtained by modifying the UVA-1 absorbance via increasing the use levels of salicylates and/or Pongamia pinnata Seed Extract, and/or by using sunscreen actives having higher UVA-1 absorbance values (e.g., at relatively lower use levels compared use levels without the synergistic combination of ingredients).

To determine whether effects on UV photoprotection in anhydrous formulation formats are similarly observed in aqueous formats, various O/W emulsions were manufactured having sunscreen actives with and without Pongamia pinnata Seed Extract at the use levels shown in Table 2, based on the total weight of the sunscreen composition. A commercially available sunscreen formulation was selected as the base, with changes to water levels accounting for the presence or absence of Pongamia pinnata Seed Extract.

TABLE 2
Example Aqueous Sunscreen Compositions

| | | Sample | Extract |
|---|---|---|---|
| Component (INCI) | | No. | (wt. %) |
| UVB Active: | Homosalate, 9.0 wt. % | 18 | 0 |
| Base: | Q.S. to 100 | 19 | 0.50 |
| UVB Active: | Octisalate, 4.5 wt. % | 20 | 0 |
| Base: | Q.S. to 100 | 21 | 0.50 |
| UVA-UVB Active: | Octocrylene, 6.0 wt. % | 22 | 0 |
| Base: | Q.S. to 100 | 23 | 0.50 |
| UVA-UVB Active: | Bemotrizinol, 3.0 wt. % | 24 | 0 |
| Base: | Q.S. to 100 | 25 | 0.50 |
| UVA Active: | Avobenzone, 3.0 wt. % | 26 | 0 |
| Base: | Q.S. to 100 | 27 | 0.50 |
| Reference: | Pongamia Pinnata Seed Extract | 28 | 0.50 |
| Base: | Q.S. to 100 | | |
| Base: | Water, Disodium EDTA, | — | — |
| | Chlorphenesin, Propylene | | |
| | Glycol, Sodium Polyacrylate, | | |
| | Xanthan Gum, Phenoxyethanol, | | |
| | Diisopropyl Adipate, | | |
| | VP/Eicosene Copolymer, | | |
| | Behenyl Alcohol, Glyceryl | | |
| | Stearate (and) PEG-100 | | |
| | Stearate, | | |
| | Acrylates/C₁₂₋₂₂ Alkyl | | |
| | Methacrylate Copolymer (and) | | |
| | Water | | |

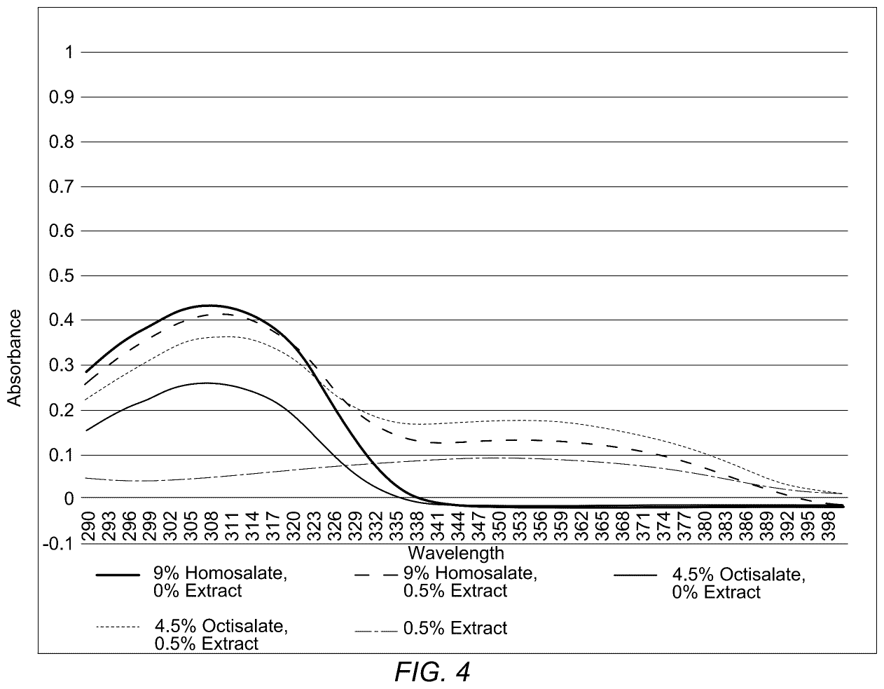

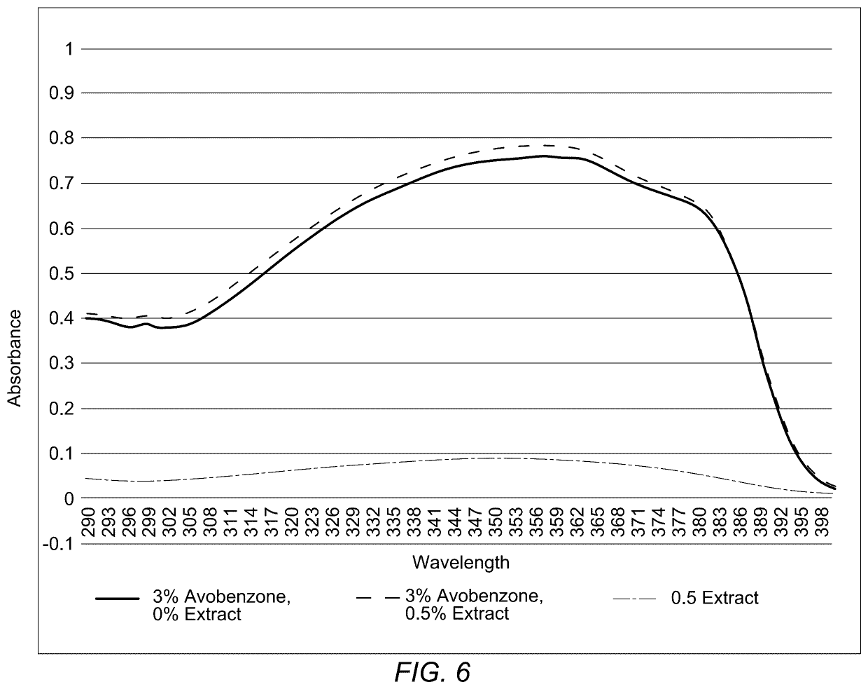

Referring to FIGS. 4-6, amplitude (vertical height) of the absorbance curves reflects the degree of photoprotection provided by the ingredients listed in Table 2. As shown in FIG. 4, an unexpected synergy in UVA-1 photoprotection is observed in aqueous sunscreen compositions including Pongamia pinnata Seed Extract and an organic sunscreen active that traditionally has no substantial photoprotection capabilities at least in the UVA-1 region. For example, the combined action from homosalate or octisalate with Pongamia pinnata Seed Extract on UVA-1 absorbance is greater than the sum of the individual effects on UVA-1 absorbance from the ingredients.

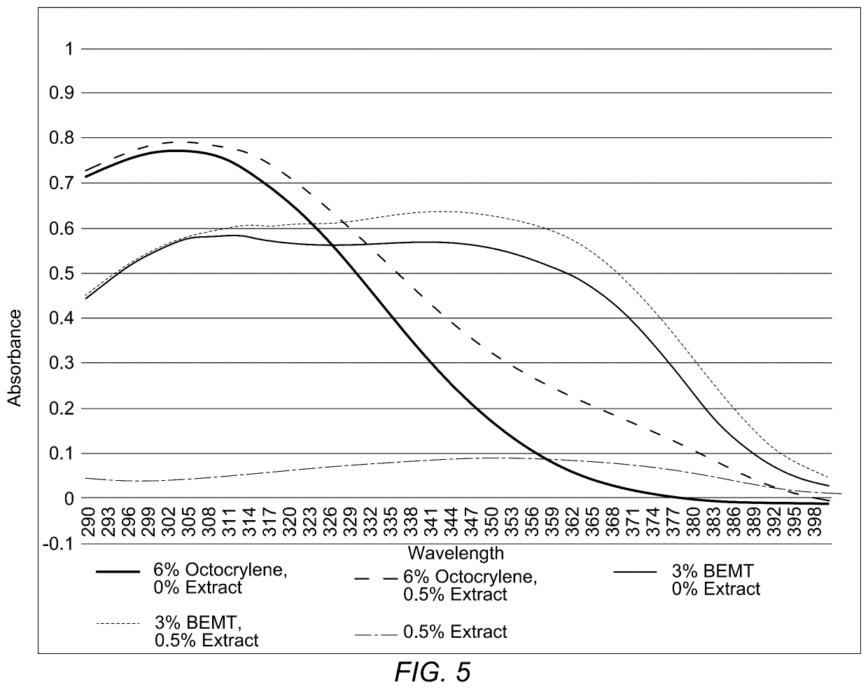

Turning now to FIG. 5, a dose dependent boost on UV photoprotection is observed in aqueous sunscreen compositions including Pongamia pinnata Seed Extract and organic sunscreen actives that traditionally have photoprotection capabilities in both the UVA region and the UVB region. As shown in FIG. 5, the combined action from octocrylene or bemotrizinol with Pongamia pinnata Seed Extract provides a dose dependent boost on UV absorbance. This contrasts with the effects on UVA photoprotection in aqueous sunscreen compositions including Pongamia pinnata Seed Extract and organic sunscreen actives that traditionally have substantial photoprotection capabilities at least in the UVA region. For example, FIG. 3 shows a substantially neutral effect in UVA photoprotection for the aqueous sunscreen compositions including Pongamia pinnata Seed Extract and avobenzone. Thus, various combinations of sunscreen actives may be incorporated at relatively lower levels for broad spectrum UV photoprotection in compositions having various formulation formats.

Additional examples include a sunscreen composition, a consumer-packaged product including a sunscreen composition, a method of using a sunscreen composition, and/or a method of making a sunscreen composition.

Additional Example 1 may include a sunscreen composition comprising at least one organic sunscreen active including a salicylate and Pongamia pinnata Seed Extract, wherein the Pongamia pinnata Seed Extract is present in an amount from about 0.1 wt. % to about 10 wt. %, based on the total weight of the sunscreen composition.

Additional Example 2 may include the sunscreen composition of Additional Example 1, wherein the Pongamia pinnata Seed Extract is present in an amount from 0.26 wt. % to 4.9 wt. %, based on the total weight of the sunscreen composition.

Additional Example 3 may include the sunscreen composition of any of Additional Examples 1-2, wherein the salicylate includes homosalate, octisalate, or combinations thereof.

Additional Example 4 may include the sunscreen composition of any of Additional Examples 1-3, wherein the salicylate is present in an amount from about 3.0 wt. % to about 20.0 wt. %, based on the total weight of the sunscreen composition.

Additional Example 5 may include the sunscreen composition of any of Additional Examples 1-4, further including one or more sunscreen actives selected from a p-aminobenzoic acid, a benzophenone, a camphor, a cinnamate, a dibenzoylmethane, an imidazole, a triazole, a triazine, a triazole, a metal oxide, or combinations thereof.

Additional Example 6 may include the sunscreen composition of any of Additional Examples 1-5, wherein the one or more sunscreen actives includes avobenzone, diethylamino hydroxybenzoyl hexyl benzoate, octocrylene, octinoxate, ethylhexyl triazone, bemotrizinol, bis-ethylhexyloxyphenol methoxyphenyl triazine, titanium dioxide, zinc oxide, or combinations thereof.

Additional Example 7 may include the sunscreen composition of any of Additional Examples 1-6, wherein the salicylate includes homosalate present in an amount of about 9.0 wt. %, based on the total weight of the sunscreen composition, and wherein the Pongamia pinnata Seed Extract is present in an amount from 0.26 wt. % to 3.0 wt. %, based on the total weight of the sunscreen composition.

Additional Example 8 may include the sunscreen composition of any of Additional Examples 1-7, wherein the salicylate includes octisalate present in an amount of about 4.5 wt. %, based on the total weight of the sunscreen composition, and wherein the Pongamia pinnata Seed Extract is present in an amount from 0.26 wt. % to 1.0 wt. %, based on the total weight of the sunscreen composition.

Additional Example 9 may include the sunscreen composition of any of Additional Examples 1-8, wherein the ratio of the salicylate to the Pongamia pinnata Seed Extract is from 2:1 to 100:1.

Additional Example 10 may include the sunscreen composition of any of Additional Examples 1-9, wherein the ratio of the salicylate to the Pongamia pinnata Seed Extract is from 4:1 to 20:1.

Additional Example 11 may include the sunscreen composition of any of Additional Examples 1-10, further including one or more auxiliary ingredients selected from carriers, film formers, surfactants, anti-aging actives, insect repellants, amino acids, colorants, pigments, dyes, opacifiers, pH adjusters, preservatives, preservative boosters, chelating agents, stabilizers, sphingolipids, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, skin cooling compounds, skin pollution protectants, skin penetration enhancers, exfoliants, staining agents, depigmenting agents, hypopigmenting agents, vitamins, antioxidants, alpha or beta hydroxy acids, retinol and its derivatives, propellants, or combinations thereof.

Additional Example 12 may include the sunscreen composition of any of Additional Examples 1-11, wherein the Pongamia pinnata Seed Extract includes pongamol and karanjin, and wherein the sunscreen composition is free of isolated pongamol, isolated karanjin, tamanu oil, Camellia sinensis leaf extract, green coffee seed extract, Angelica archangelica root extract, citrus aurantium peel extract, mica, ruby, titanium dioxide, zinc oxide, polyesters, trimethoxybenzylidene pentanedione, penthaerythrityl tetra-di-t-butyl hydroxyhydrocinnamate, or combinations thereof.

Additional Example 13 may include the sunscreen composition of any of Additional Examples 1-12, wherein the sunscreen composition is free of a metal oxide in an amount effective to provide a sun protection factor of at least 8.

Additional Example 14 may include the sunscreen composition of any of Additional Examples 1-13, wherein the sunscreen composition is in the form of an anhydrous composition.

Additional Example 15 may include the sunscreen composition of any of Additional Examples 1-14, wherein the sunscreen composition is in the form of an aqueous composition.

Additional Example 16 may include a sunscreen composition comprising at least one organic UVB sunscreen active selected from the group consisting of salicylates having no substantial photoprotection capabilities within the Ultraviolet A-1 (UVA-1) region of the electromagnetic spectrum, and Pongamia pinnata Seed Extract present in an amount from 0.26 wt. % to 4.9 wt. %, based on the total weight of the sunscreen composition, wherein the sunscreen composition is free of isolated pongamol, isolated karanjin, tamanu oil, Camellia sinensis leaf extract, green coffee seed extract, Angelica archangelica root extract, citrus aurantium peel extract, mica, ruby, titanium dioxide, zinc oxide, polyesters, trimethoxybenzylidene pentanedione, penthaerythrityl tetra-di-t-butyl hydroxyhydrocinnamate, or combinations thereof.

Additional Example 17 may include the sunscreen composition of Additional Example 16, wherein the ratio of the salicylates to the Pongamia pinnata Seed Extract is from 4:1 to 20:1.

Additional Example 18 may include a consumer-packaged product comprising a container and the sunscreen composition of any of Additional Examples 1-17 housed in the container.

Additional Example 19 may include a method to protect a keratinous surface from UV radiation comprising contacting the keratinous surface with the sunscreen composition of any of Additional Examples 1-17.

Additional Example 20 may include a method to make the sunscreen composition of any of Additional Examples 1-17 comprising selecting at least one organic sunscreen active, selecting Pongamia pinnata Seed Extract, and mixing the at least one organic sunscreen active and the Pongamia pinnata Seed Extract.

Additional Example 21 may include the method of Additional Example 20, wherein selecting the at least one organic sunscreen active comprises identifying at least one organic sunscreen active having no substantial photoprotection capabilities within the UVA-1 region of the electromagnetic spectrum, and mixing the Pongamia pinnata Seed Extract with the at least one organic sunscreen active for a synergistic increase in the sunscreen composition to at least the UVA-1 absorbance, the critical wavelength, the UVA-1/UV ratio, or combinations thereof.

In this disclosure and the following claims, the word “or” indicates an inclusive list such that, for example, the list of X, Y, or Z means X or Y or Z or XY or XZ or YZ or XYZ. Similarly, the list of one or more of X, Y, or Z or a list of X, Y, Z, or combinations thereof means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

While the present disclosure has been particularly described, in conjunction with specific preferred embodiments, it is evident that many alternatives, modifications, and variations will be apparent to those skilled in the art in light of the foregoing description. It is therefore contemplated that the appended claims will embrace any such alternatives, modifications, and variations as falling within the true scope and spirit of the present disclosure.

What is claimed is:

1. A sunscreen composition comprising:
at least one organic sunscreen active including a salicylate; and
Pongamia pinnata Seed Extract, wherein the Pongamia pinnata Seed Extract is present in an amount from 0.26 wt. % to 4.9 wt. %, based on the total weight of the sunscreen composition.

2. The sunscreen composition of claim 1, wherein the salicylate includes homosalate, octisalate, or combinations thereof.

3. The sunscreen composition of claim 1, wherein the salicylate is present in an amount from about 3.0 wt. % to about 20.0 wt. %, based on the total weight of the sunscreen composition.

4. The sunscreen composition of claim 1, further including one or more sunscreen actives selected from a p-aminobenzoic acid, a benzophenone, a camphor, a cinnamate, a dibenzoylmethane, an imidazole, a triazole, a triazine, a triazole, a metal oxide, or combinations thereof.

5. The sunscreen composition of claim 4, wherein the one or more sunscreen actives includes avobenzone, diethylamino hydroxybenzoyl hexyl benzoate, octocrylene, octinoxate, ethylhexyl triazone, bemotrizinol, bis-ethylhexyloxyphenol methoxyphenyl triazine, titanium dioxide, zinc oxide, or combinations thereof.

6. The sunscreen composition of claim 1, wherein the salicylate includes homosalate present in an amount of about 9.0 wt. %, based on the total weight of the sunscreen composition, and wherein the Pongamia pinnata Seed Extract is present in an amount from 0.26 wt. % to 3.0 wt. %, based on the total weight of the sunscreen composition.

7. The sunscreen composition of claim 1, wherein the salicylate includes octisalate present in an amount of about 4.5 wt. %, based on the total weight of the sunscreen composition, and wherein the Pongamia pinnata Seed Extract is present in an amount from 0.26 wt. % to 1.0 wt. %, based on the total weight of the sunscreen composition.

8. The sunscreen composition of claim 1, wherein the ratio of the salicylate to the Pongamia pinnata Seed Extract is from 2:1 to 100:1.

9. The sunscreen composition of claim 8, wherein the ratio of the salicylate to the Pongamia pinnata Seed Extract is from 4:1 to 20:1.

10. The sunscreen composition of claim 1, further including one or more auxiliary ingredients selected from carriers, film formers, surfactants, anti-aging actives, insect repellants, amino acids, colorants, pigments, dyes, opacifiers, pH adjusters, preservatives, preservative boosters, chelating agents, stabilizers, sphingolipids, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, skin cooling compounds, skin pollution protectants, skin penetration enhancers, exfoliants, staining agents, depigmenting agents, hypopigmenting agents, vitamins, antioxidants, alpha or beta hydroxy acids, retinol and its derivatives, propellants, or combinations thereof.

11. The sunscreen composition of claim 1, wherein the Pongamia pinnata Seed Extract includes pongamol and karanjin, and wherein the sunscreen composition is free of isolated pongamol, isolated karanjin, tamanu oil, Camellia sinensis leaf extract, green coffee seed extract, Angelica archangelica root extract, citrus aurantium peel extract, mica, ruby, titanium dioxide, zinc oxide, polyesters, trimethoxybenzylidene pentanedione, penthaerythrityl tetra-di-t-butyl hydroxyhydrocinnamate, or combinations thereof.

12. The sunscreen composition of claim 1, wherein the sunscreen composition is free of a metal oxide in an amount effective to provide a sun protection factor of at least 8.

13. The sunscreen composition of claim 1, wherein the sunscreen composition is in the form of an anhydrous composition.

14. The sunscreen composition of claim 1, wherein the sunscreen composition is in the form of an aqueous composition.

15. A sunscreen composition comprising:
at least one organic UVB sunscreen active selected from the group consisting of salicylates having no substantial photoprotection capabilities within the Ultraviolet A-1 (UVA-1) region of the electromagnetic spectrum; and
Pongamia pinnata Seed Extract present in an amount from 0.26 wt. % to 4.9 wt. %, based on the total weight of the sunscreen composition, wherein the sunscreen composition is free of isolated pongamol, isolated karanjin, tamanu oil, Camellia sinensis leaf extract, green coffee seed extract, Angelica archangelica root extract, citrus aurantium peel extract, mica, ruby, titanium dioxide, zinc oxide, polyesters, trimethoxybenzylidene pentanedione, penthaerythrityl tetra-di-t-butyl hydroxyhydrocinnamate, or combinations thereof.

16. The sunscreen composition of claim 15, wherein the ratio of the salicylates to the Pongamia pinnata Seed Extract is from 4:1 to 20:1.

17. A consumer-packaged product comprising:
a container; and
the sunscreen composition of claim 1 housed in the container.
